(19) United States
(12) Patent Application Publication
(10) Pub. No.: US 2004/0254142 A1
(43) Pub. Date: Dec. 16, 2004

(54) Arthritic and joint condition formulation

(76) Inventor: Robert Kovler, Brooklyn, NY (US)

Correspondence Address: GOTTLIEB RACKMAN & REISMAN PC, 270 MADISON AVENUE, 8TH FLOOR, NEW YORK, NY, 100160601

(21) Appl. No.: 10/635,229

(22) Filed: Aug. 4, 2003

Related U.S. Application Data
(60) Provisional application No. 60/478,506, filed on Jun. 12, 2003.

(51) Int. Cl. A61K 31/737; A61K 31/7008

(52) U.S. Cl. 514/54; 514/62

(57) ABSTRACT

An improved arthritic and joint condition formulation is provided. The formulation includes glucosamine and salts thereof, chondroitin and salts thereof, and one of manganese chelate and a carotenoid.

### BACKGROUND OF THE INVENTION

This invention relates to an arthritic and joint condition formulation, and more particularly to an arthritic and joint condition formulation utilizing glucosamine and chondroitin in the treatment of arthritic joint conditions in dogs and other mammals.

The connective tissues of mammals are often subjected to stresses and strains which can result in afflictions, such as arthritis, joint inflammation and stiffness. Such afflictions are not only painful, but are often debilitating. Steroids, such as corticosteroids and other anti-inflammatories, are often used for the treatment of joint and arthritic afflictions. Although such materials often relieve the pain and swelling associated with connective tissue problems, these types of drugs eventually wear out in their effectiveness. Furthermore, these types of drugs may also inhibit the body's own natural healing processes, leading to further deterioration of the connective tissue.

U.S. Pat. No. 5,364,845 describes a therapeutic composition for the protection, treatment and repair of connective tissue in mammals. The composition includes glucosamine and preferably chondroitin sulfate. The composition further includes manganese ascorbate, which acts as a catalyst in the absorption and the production of both proteoglycens and collagens. While this composition, and its method of use, is effective, especially in stimulating collagen and proteoglycen synthesis, it is less than desirable. This is because bio-absorption is not sufficiently adequate.

Accordingly, it is necessary to provide a therapeutic composition which includes glucosamine, chondroitin and either a form of manganese that is bio-available, or a carotenoid such as astaxanthin.

### SUMMARY OF THE INVENTION

Generally speaking, and in accordance with the invention, an improved arthritic and joint condition formulation is provided. The formulation includes glucosamine and salts thereof, chondroitin and salts thereof, and at least one of manganese chelate and a carotenoid. The formulation is typically mixed with water and is delivered orally to the mammal. The provision of manganese chelate provides for improved bioavailability of the manganese when the inventive formulation is ingested. Manganese chelate is extremely soluble in the intestines, thus manganese absorption is enhanced by the use of manganese chelates. The provision of a carotenoid helps protect the body by decreasing and repairing damage to cells and tissues.

Accordingly, it is an object of the invention to provide an improved arthritic and joint condition formulation.

It is another object of the invention to provide an improved arthritic and joint condition formulation which contains only naturally occurring components capable of providing beneficial therapeutic effects.

Yet another object of the invention is to provide an improved arthritic and joint condition formulation which exhibits increased absorption rates.

Still other objects and advantages of the invention will in part be obvious and will in part be apparent from the following description.

### DETAILED DESCRIPTION

The inventive formulation includes glucosamine [and/or salts thereof], preferably in an amount between about 2.0 and 55.0 weight percent in the formulation. The preferred glucosamine is the salt glucosamine hydrochloride. Also usable is the salt glucosamine sulphate. In salt form, delivery and uptake by the animal is enhanced. The glucosamine ingredient has the purpose of helping to build cartilage between joints in the mammal that is treated. Indeed, glucosamine is one of the biological chemicals that forms all of the major cushioning ingredients of the joint fluids and surrounding tissue. Glucosamine helps to make the synovial thick and elastic, in joints and vertebrae.

The inventive formulation also includes chondroitin [and/or salts thereof], preferably in an amount between about 1.5 and 36.5 weight percent in the formulation. The preferred chondroitin is the salt chondroitin sulphate. The purpose of the chondroitin component is to block the destructive enzymes that breakdown cartilage in the joints. Significantly, chondroitin is also one of the products necessary for the synthesis of glycosaminoglycans, but plays a more important role here by combating and neutralizing destructive enzymes in the joints. Thus, by adding chondroitin to the inventive formulation, the level of these destructive enzymes is reduced.

The formulation must also include at least on of manganese chelate and a carotenoid. For manganese chelate, it is preferably included in the formulation in an amount between about 0.03 and 0.85 weight percent. The purpose of including manganese chelate is as follows. An amino acid forms something called mineral chelates. Mineral chelates have excellent bioavailability. A mineral must be in a form that is usable by the animal's body as in the process of chelation. In order for manganese to be bioavailable, it must be soluble in the intestines. The intestinal walls will not absorb large particles and thus the formulation is preferably in a liquid form. A chelated form of manganese will happen when the central atom is attached to cause a reaction with another molecule in order to form a ring type structure. The better absorption will help with bone development and the metabolism of carbohydrates. In other words, manganese chelate has excellent absorption of manganese into the animal's system. Improved manganese absorption in turn improves animal bone growth. Manganese is also an excellent antioxidant.

The use of manganese chelate is superior to the prior art teaching of manganese ascorbate in that it has improved absorption and improved bio-availability.

A carotenoid, if included in the inventive formulation, will be present preferably in an amount between 0.02 and 0.90 weight percent. The preferred carotenoid is astaxanthin, a fat soluble anti-oxidant, which typically requires a surfactant in order to be mixed into water. Other suitable carotenoid include vitamin E, beta carotene, zeaxanthin, lutein, and canthaxanthin. Carotenoids are anti-oxidants and are important in the fighting of free radicals. Indeed, oxidative damage plays a significant role in advancement of degenerative diseases. Carotenoid antioxidants are important for the following reason. Oxygen, taken in when you breathe, causes a chemical reaction called oxidation in your cells as a by-product of normal metabolism. This process results in the formation of molecules known as free radicals, which can damage cells and tissues. If left unchecked, damage may result to the cardiovascular disease, or certain cancers and other degenerative diseases like osteoarthritis may occur. Carotenoid supplementation can slow the formation of free radicals, protecting the body by decreasing and repairing the damage to cells and tissues.

The inventive formulation may also include one or more anti-inflammatories, preferably in a range amount of between about 2.0 and 55.0 weight percent. For example, the formulation may include aloe barbadenis (aloe vera). The purpose of including aloe is to achieve an anti-inflammatory effect as well as to reduce gastrointestinal problems. As a further example, the inventive formulation may also include methyl sulfonyl methane (msm), which constitutes a sulphur anti-inflammatory and serves the purpose of an anti-inflammatory. Another anti-inflammatory is cayenne and its derivatives, including capsicum and capsaicin.

The inventive formulation may also include the anti-oxidant calcium ascorbate (vitamin c), preferably in an amount between 0.1 and 10.6 weight percent. Calcium ascorbate helps strengthen the immune system and supports joint and connective tissues. Calcium ascorbate such as the patented form “Ester-C,” is rapidly absorbed and non-acidic to the stomach. Ascorbic acid could also be used.

The inventive formulation may also include a ph balancing agent such as citric acid, preferably in an amount between 0.04 and 0.20 weight percent.

The inventive formulation also includes preservatives such as potassium sorbate and sodium benzoate in an amount between about 0.05 and 0.75 weight percent. The purpose of using such preservatives is to prevent the formulation from becoming rancid or collecting fungus.

The inventive formulation may also include an emulsifier such as glycerin and other well known emulsifiers, preferably in an amount between about 4.0 and 80 weight percent. The purpose of including an emulsifier is to emulsify the anti-oxidant and to prevent it from freezing in cold weather.

The inventive formulation may also include one or more amino acids, preferably in an amount 0.02 and 0.18 weight percent. Such amino acids are selected from Taurine, Alanine, Arginine, Aspartic, Cysteine, Glutamic Acid, Glycine, Histidine, Leucine, Proline, Serine and Tyrosine.

The inventive formulation may also include some type of flavoring, preferably in an amount between 0.1 and 0.7 weight percent. The purpose of including a flavoring in the inventive formulation is to make it palatable. Examples of flavoring include prime rib, chicken, liver, beef, salmon and other fish flavors.

The inventive formulation may also include food additives, by way of “Stevia 90%” (a natural sweetener) and “Tween 80” (polyoxyethylene sorbitan monooleate).

An example of the inventive formulation, after being mixed with water, is as follows:

| | |
|---|---|
| Glucosamine HCl | 750 mg. per 15 ml. |
| Methyl Sulfonyl Methane | 750 mg. per 15 ml. |
| Chondroitin Sulphate | 500 mg. per 15 ml. |
| Aloe Vera | 300 mg. per 15 ml. |
| Ester-C (Calcium Ascorbate) | 175 mg. per 15 ml. |
| Manganese Chelate | 10 mg. per 15 ml. |
| Astaxanthin | 1 mg. per 15 ml. |

It is noted that the inventive formulation is normally mixed in water before oral delivery to a mammal. More or less water is added depending on the concentration that is desired.

In one example. the inventive formulation is prepared as follows. A suitable amount of glucosamine, chondroitin, manganese chelate, ester-C, citric acid, glycerin and stevia are dissolved in water by gentle warming. Then, preservatives, such as sodium benzoate and potassium sorbate may be mixed therein to produce a solution. Then a suitable amount of astaxanthin, preferably mixed with a suitable surfactant, is added to the solution in order to produce the inventive formulation in a liquid form.

In use, the inventive formulation, when mixed with water, constitutes a liquid, and is typically mixed with food and taken at a dosage level per day as follows:

| | |
|---|---|
| Under 10 lbs. | ¼ oz. |
| 10-24 lbs. | ½ oz. |
| 25-49 lbs. | 1 oz. |
| 50-100 lbs. | 1 ¼ oz. |
| 100 lbs. | 1 ½ oz |

It will thus be seen that the objects set forth above, among those made apparent from the preceding description, are efficiently attained and, since certain changes may be made in the construction of the inventive formulation without departing from the spirit and scope of the invention, it is intended that all matter contained in the above description as shown in the accompanying drawings shall be interpreted as illustrative and not in a limiting sense.

It is also to be understood that the following claims are intended to cover all the generic and specific features of the invention herein described and all statements of the scope of the invention, which as a matter of language, might be said to fall therebetween.

1. A therapeutic composition for the treatment of arthritic and joint conditions comprising a therapeutic quantity of glucosamine and/or salts thereof, a therapeutic quantity of chondroitin and/or the salts thereof, and a therapeutic quantity of a compound selected from the group consisting of manganese chelate and a carotenoid.

2. The composition of claim 1, wherein glucosamine and/or the salts thereof are present in an amount between about 2 and 55 weight percent, and wherein chondroitin and/or the salts thereof are present in an amount between about 1.5 and 36.5 weight percent.

3. The composition of claim 2, wherein said compound is manganese chelate in an amount between about 0.03 and 0.85 weight percent.

4. The composition of claim 2, wherein glucosamine and/or salts thereof is selected from the group consisting of glucosamine hydrochloride and glucosamine sulphate.

5. The composition of claim 4, wherein glucosamine and/or salts thereof comprises glucosamine hydrochloride.

6. The composition of claim 2, wherein chondroitin and/or salts thereof comprises chondroitin sulphate.

7. The composition of claim 2, wherein said compound is a carotenoid in an amount between about 0.02 and 0.90 weight percent.

8. The composition of claim 7, wherein the carotenoid is selected from the group consisting of astaxanthin, vitamin E, beta carotene, zeaxanthin, lutein and canthaxanthin.

9. The composition of claim 2, further including an anti-inflammatory in an amount between about 2.0 and 55.0 weight percent.

10. The composition of claim 9, wherein the anti-inflammatory is selected from the group consisting of aloe barbadenis, methyl sulfonyl methane, cayennne, capsium and capsaicin.

11. The composition of claim 10, wherein the anti-inflammatory comprises both aloe barbadenis and methyl sulfonyl methane.

12. The composition of claim 2, further including calcium ascorbate in an amount between about 0.1 and 10.6 weight percent.

13. The composition of claim 2, further including a ph balancing agent in an amount between about 0.04 and 0.20.

14. The composition of claim 13, wherein the ph balancing agent comprises citric acid.

15. The composition of claim 2, further including one or more preservatives in an amount between about 0.05 and 0.75 weight percent.

16. The composition of claim 15, wherein said preservatives are selected from the group consisting of potassium ascorbate and sodium benzoate.

17. The composition of claim 2, further comprising an emulsifier in an amount between about 4.0 and 80 weight percent.

18. The composition of claim 2, further comprising an emulsifier in an amount between about 4.0 and 80 weight percent.

19. The composition of claim 18, wherein the emulsifier comprises glycerin.

20. The composition of claim 2, further including one or more amino acids in an amount 0.02 and 0.18 weight percent.

21. The composition of claim 20, wherein said amino acids are selected from the group consisting of Taurine, Alanine, Arginine, Aspartic, Cysteine, Glutamic Acid, Glycine, Histidine, Leucine, Proline, Serine and Tyrosine.

22. The composition of claim 2, further including at least one of a flavoring and a food additive.

23. The composition of claim 2, wherein said compound comprises manganese chelate in an amount between about 0.03 and 0.85 weight percent and a carotenoid in an amount between 0.02 and 0.90 weight percent.

24. A method for treating arthritic and joint conditions in an animal comprising the steps of administering a therapeutically effective quantity of glucosamine and/or the salts thereof, a therapeutically effective quantity of chondroitin and/or the salts thereof, and a therapeutically effective quantity of a compound selected from the group consisting of manganese chelate and a carotenoid.

25. The method of claim 24, wherein said compound is manganese chelate.

26. The method of claim 24, wherein said compound is carotenoid.
